(19) United States
(12) Patent Application Publication
(10) Pub. No.: US 2015/0232952 A1
(43) Pub. Date: Aug. 20, 2015

(54) QUANTITATIVE GENETIC ANALYSIS OF ARTICLES INCLUDING GOSSYPIUM BARBADENSE AND GOSSYPIUM HIRSUTUM COTTON

(71) Applicant: APDN (B.V.I) INC., Tortola (VG)

(72) Inventors: Yuhua Sun, Stony Brook, NY (US); MingHwa Benjamin Liang, East Setauket, NY (US); Xiaoqian Jin, East Setauket, NY (US)

(21) Appl. No.: 14/631,992

(22) Filed: Feb. 26, 2015

Related U.S. Application Data
(63) Continuation-in-part of application No. 14/191,947, filed on Feb. 27, 2014.
Application No. 14/191,947 is a continuation of application No. 12/269,737, filed on Nov. 12, 2008, now Pat. No. 8,669,079.
Application No. 12/269,737 is a continuation-in-part of application No. 14/584,309, filed on Dec. 29, 2014.
Application No. 14/584,309 is a continuation of application No. 12/269,757, filed on Nov. 12, 2008, now Pat. No. 8,940,485.

(51) Int. Cl. C12Q 1/68

(52) U.S. Cl. CPC C12Q 1/6895; C12Q 2600/13; C12Q 2600/16

(57) ABSTRACT

Exemplary embodiments of the present invention provide a method for assessing a proportion of one or more cotton species in an article comprising cotton. The method includes providing a sample including cotton fibers from the article comprising cotton. Cotton DNA is extracted from the cotton fibers to provide extracted cotton DNA. The extracted cotton DNA is analyzed to identify a presence of one or more cotton species included in the article comprising cotton. Proportions of the one or more cotton species included in the article comprising cotton are assessed.

### RELATED APPLICATIONS

This application is a continuation in part of U.S. patent application Ser. No. 14/191,947, filed Feb. 27, 2014, which is a continuation of U.S. patent application Ser. No. 12/269,737, filed on Nov. 12, 2008, which issued as U.S. Pat. No. 8,669,079 on Mar. 11, 2014; this application is also a continuation in part of U.S. patent application Ser. No. 14/584,309, filed Dec. 29, 2014, which is a continuation of U.S. patent application Ser. No. 12/269,757, filed on Nov. 12, 2008, which issued as U.S. Pat. No. 8,940,485 on Jan. 27, 2015, the entire disclosures of each of which are incorporated by reference herein in their entireties.

### FIELD OF THE INVENTION

The present invention relate to methods of quantitative genetic analysis of one or more cotton species. The methods according to the present invention identify one or more species of cotton included in an article, such as a textile article. The cotton species identified may be G. barbadense and/or G. hirsutum cotton or any other cotton species having an identifiable nucleic acid sequence polymorphism. The methods according to the present invention provide quantitative genetic analysis of an article, such as textile article, including G. barbadense and/or G. hirsutum cotton to determine a proportion of each cotton species included in the article.

### BACKGROUND

Cotton is an essential cash crop throughout the United States and the world. Cotton is particularly important in forming a variety of goods, for example, fabrics, clothing and household items such as towels, curtains and tablecloths. The use of cotton to generate fabric generally involves processing of bales of cotton to liberate cotton fibers. Bales of cotton are frequently handled by automated machinery to remove unprocessed lint. The lint can then be cleaned by, for example, using a blower to separate short components of the lint from cotton fibers. The separated cotton fibers can then be woven into longer strands sometimes referred to as yarn or cotton yarn. A single pound of cotton may yield many millions of cotton fibers. However, the lengths of cotton fibers vary according to the species or cultivars of the cotton plant from which the fibers are derived.

Generally, two species of cotton are commercially cultivated throughout the world, namely Gossypium barbadense (G. barbadense) and Gossypium hirsutum (G. hirsutum). G. barbadense produces relatively long fibers, which may be referred to as extra long staple (ELS) cotton fibers. G. hirsutum produces relatively short fibers, which are often referred to as Upland cotton fibers. Many regions around the world produce ELS cotton with distinct fiber qualities, such as Egyptian Pima and Indian Pima. ELS cotton is generally considered to produce higher quality and therefore higher value fabrics, clothing, household items, and related products. Types of ELS cotton include, for example, American Pima, Egyptian, and Indian Suvin. Branded products carrying a particular ELS label, such as American Pima, Egyptian, Supima, or Indian Suvin labels will generally command a higher price than products lacking such a designation. Thus, detection of authentic ELS cotton products is critical for textile manufacturers and distributors at all stages of the global supply chain to eliminate adulterated ELS cotton that includes a percentage of Upland cotton or other non-ELS cotton.

Despite a common ancestry, over time, isolation and/or cross breeding of cotton species has created subtle but unique genetic variations in cultivars from different regions. Even though ELS cotton cultivars belong to the species G., barbadense, ELS cotton cultivars from certain regions, such as American Pima, have superior fiber qualities compared to ELS cotton cultivars grown in other regions of the world and are heavily promoted and highly sought after by textile manufacturers. Thus, ELS cotton from certain geographic regions is more valuable than ELS cotton from other regions or non-ELS cotton. Articles such as textile articles manufactured from ELS fibers are considered of higher quality as compared to those made of G. hirsutum fibers (Upland). Traditionally, the distinction between G. barbadense and G. hirsutum fibers is made by comparing many aspects of fiber physical qualities such as fiber length, strength, and uniformity. However, it is difficult if not impossible to distinguish between raw and processed cotton fibers produced from these two species, let alone the proportion of each type of fiber included in an article including a blend of cotton fibers two or more species of cotton. Once raw cotton fibers are processed and spun into yarn, or are ultimately woven into textiles or fabrics, most physical properties of the raw cotton fibers are altered, and there is no reliable method to determine the origin or species of the cotton fibers included in the yarn or textile(s).

Producing counterfeit clothing products or producing knock-off textile items is a serious problem for the textile industry, costing manufacturers and retail stores millions of dollars annually in the United States alone. Raw cotton or bales of raw cotton may be imported or exported from one region or country to another region or county and identifying the presence and/or proportions of species of cotton included in the bale of raw cotton may be desirable. Being able to identify the species of cotton and proportions of cotton fibers utilized in a textile article would not only be a way to authenticate an item as legitimate, but would also enable the detection of forged or counterfeit textile products.

### SUMMARY

Exemplary embodiments of the present invention provide methods for assessing a proportion of one or more cotton species in an article including cotton. The methods include providing a sample including cotton fibers, such as mature cotton fibers, from the article including cotton. Cotton DNA is extracted from the cotton fibers to provide extracted cotton DNA. The extracted cotton DNA is analyzed to identify a presence of one or more cotton species included in the article including cotton. Proportions of the one or more cotton species included in the article including cotton are assessed. For example and without limitation, the extracted DNA includes chloroplast DNA. The extracted DNA might include nuclear DNA and/or mitochondrial DNA in addition to chloroplast DNA. The one or more cotton species include G. barbadense and/or G. hirsutum.

Analyzing the extracted DNA may include a polymerase chain reaction (PCR). Analyzing the extracted cotton DNA may include amplifying the extracted cotton DNA using at least one set of specific primers complementary to a non-variable region of the one or more cotton species.

The one or more cotton species may be identified by using one or more hybridization probes. Each of the hybridization probes is complementary to a variable region sequence specific to a cotton species of the one or more cotton species. The variable region includes a DNA sequence which is not conserved between the cotton species. Each of the hybridization probes may include a detectable marker, such as a fluorescent marker. The sequence specific to the cotton species of the one or more cotton species may include a sequence polymorphism between a first cotton species and a second cotton species of the one or more cotton species. The sequence polymorphism between the first cotton species and the second cotton species might include a sequence polymorphism, a sequence length polymorphism or both a sequence polymorphism and a sequence length polymorphism. The variable region sequence specific to the cotton species of the one or more cotton species may be in a variable region of G. barbadense. Alternatively, the variable region sequence specific to the cotton species of the one or more cotton species may be in a variable region of G. hirsutum.

Exemplary embodiments of the present invention provide methods for assessing a proportion of one or more cotton species in an article including cotton. The method includes providing a sample including cotton fibers from the article including cotton. Cotton DNA is extracted from the cotton fibers to provide extracted cotton DNA. A portion of the extracted cotton DNA is amplified by qPCR and one or more amplified products are produced. The one or more amplified products are analyzed to identify a presence of at least one cotton species in the cotton fibers from the article including cotton. A threshold cycle number is determined for the extracted cotton DNA. The threshold cycle number for the extracted DNA is compared to a known threshold cycle number. Proportions of the one or more cotton species included in the article including cotton are assessed.

Exemplary embodiments of the present invention provide methods for assessing a proportion of one or more cotton species in an article including cotton. The method includes providing a sample including cotton fibers from the article including cotton. Cotton DNA is extracted from the cotton fibers to provide extracted cotton DNA. A portion of the extracted cotton DNA is amplified by qPCR and one or more amplified products are produced. The one or more amplified products are analyzed to identify a presence of a first cotton species and/or a second cotton species in the cotton fibers from the article including cotton. A threshold cycle number for the extracted DNA of the first cotton species is determined. A threshold cycle number for the extracted DNA of the second cotton species is determined. The first and second threshold cycle numbers are compared to each other. Proportions of the first cotton species and the second cotton included in the article including cotton are assessed. The extracted DNA may be amplified by multiplex qPCR.

### BRIEF DESCRIPTION OF THE FIGURES

FIG. 1 is a diagram illustrating variable and non-variable regions of ELS and non-ELS cotton species.

FIG. 2 is a flow chart illustrating a method of assessing a proportion of one or more cotton species included in an article including cotton according to an exemplary embodiment of the present invention.

FIG. 3 is a flow chart illustrating a method of assessing a proportion of one or more cotton species included in an article including cotton according to an exemplary embodiment of the present invention.

FIG. 4 is a multiplex qPCR amplification curve for a sample from a textile article including 100% ELS cotton.

FIG. 5 is a multiplex qPCR amplification curve for a sample from a textile article including 100% non-ELS cotton.

FIG. 6 is a graph illustrating experimentally determined proportions of ELS cotton included in an article including cotton compared with known proportions of ELS cotton included in the article including cotton.

FIG. 7 is a graph illustrating experimentally determined proportions of non-ELS cotton included in an article including cotton compared with known proportions of ELS cotton included in the article including cotton.

FIG. 8 illustrates multiplex qPCR amplification curves showing threshold cycle numbers for ELS and Upland cotton included in a textile article including a blend of ELS and Upland cotton.

FIG. 9 illustrates multiplex qPCR amplification curves showing threshold cycle numbers for ELS and Upland cotton included in a textile article including a blend of ELS and Upland cotton.

FIG. 10 illustrates multiplex qPCR amplification curves showing threshold cycle numbers for ELS and Upland cotton included in a textile article including a blend of ELS and Upland cotton.

### DETAILED DESCRIPTION

Exemplary embodiments of the present invention provide methods of quantitative genetic analysis of one or more cotton species. The methods provide a method for definitive identification of articles, such as textile articles, including G. barbadense and/or G. hirsutum cotton. In particular, the methods provide quantitative genetic analysis of articles, such as textile articles, including G. barbadense and/or G. hirsutum cotton to determine a proportion of each cotton species included in the article.

### DEFINITIONS

The term “ELS” means “extra long staple” cotton fibers. For example, those fibers produced from G. barbadense are ELS cotton fibers.

The term “upland fiber” defines cotton fibers which are shorter than ELS cotton fibers. For example, upland fibers are produced from G. hirsutum.

The term “variable region” means a genetic region of similar cotton species which has sequence variations between species. That is, variable regions between cotton species include one or more sequence or length polymorphisms, which are not conserved between species. The variation may be, for example, a difference in sequence length within a genetic region, or single nucleotide changes within a specific genetic region. Variable regions may exist between cotton species in nuclear DNA, mitochondrial DNA or chloroplast DNA of the cotton species.

The term “primer” means an oligonucleotide with a specific nucleotide sequence which is sufficiently complimentary to a particular sequence of a target DNA molecule, such that the primer specifically hybridizes to the target DNA sequence.

The term “probe” refers to a binding component which binds preferentially to one or more targets (e.g., antigenic epitopes, polynucleotide sequences, macromolecular receptors) with an affinity sufficient to permit discrimination of labeled probe bound to a target from a nonspecifically bound labeled probe (i.e., background).

The term “PCR” means polymerase chain reaction. This refers to any technology where a nucleotide sequence is amplified via temperature cycling techniques in the presence of a nucleotide polymerase, preferably a thermostable DNA polymerase. This includes but is not limited to real-time PCR technology, reverse transcriptase-PCR, and standard PCR methods.

In general, the term “textile” may be used to refer to fibers, yarns, or fabrics. More particularly, the term “textile” as used herein refers to raw cotton, ginned cotton, cotton fibers, cotton yarns, cotton fabrics, yarn that is blended with cotton, fabric that is blended with cotton, or any combination thereof.

The term “mature cotton fiber” as used herein refers to a cotton fiber wherein a lumen wall that separates the secondary wall (consisting of cellulose) from the lumen that has naturally collapsed. The lumen is the hollow canal that runs the length of the fiber and is filled with living protoplast during the growth period; after the fiber matures and the boll opens the protoplast dries up and the lumen will naturally collapse and leave a large central void in each fiber.

The genomes of G. barbadense cotton and G. hirsutum cotton are highly conserved between species. However, there are detectable genetic variations in variable regions (i.e., non-conserved regions) between G. barbadense and G. hirsutum, which may be utilized in identifying one or more of the cotton species and distinguishing one cotton species from another. For example, variable regions in the chloroplast DNA of G. barbadense and G. hirsutum cotton include a number of sequence polymorphisms and sequence length polymorphisms between species. One or more sequence polymorphisms and/or sequence length polymorphisms may be used to identify the presence of one or more species, and quantitatively assess a proportion of each cotton species included in an article, such as a textile article. An absence of a fluorescent signal generated by one or more hybridization probes may be used to determine an absence of one or more cotton species in an article.

While some of the exemplary embodiments of the present invention describe quantitative analysis of articles including one or two species of cotton (e.g., G. barbadense and/or G. hirsutum), exemplary embodiments of the present invention are not limited thereto or thereby. The present invention may similarly be used to qualitatively or quantitatively analyze articles including more than two species of cotton. For example, a sample including cotton fibers may include cotton fibers of a third cotton species (e.g., G. arboretum) and/or a fourth cotton species (e.g., G. herbaceum). That is, an article such as a textile article may include a blend of more than two cotton species, and a sample obtained from the article may include cotton fibers of each of the cotton species included in the article.

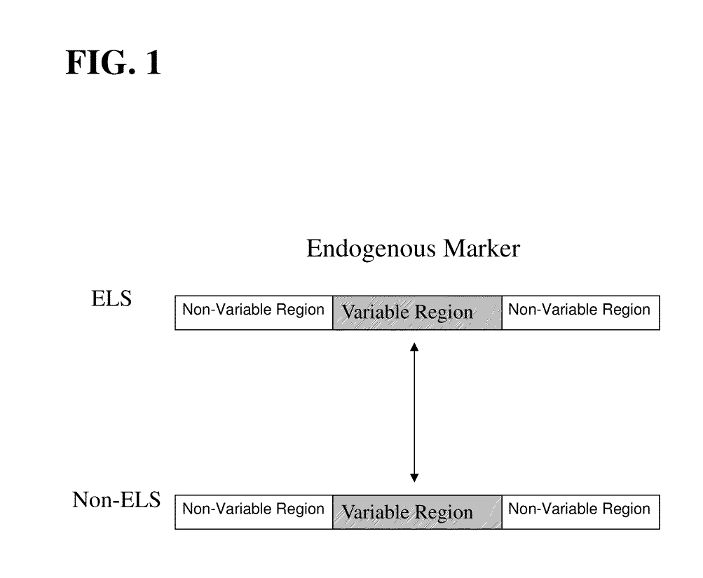

FIG. 1 is a diagram illustrating variable and non-variable regions of ELS and non-ELS cotton species. FIG. 1 illustrates a variable region of an Extra Long Staple (ELS) cotton species (e.g. G. barbadense) and a Non-ELS (Upland) cotton species (e.g. G. hirsutum). The variable region may differ in either sequence length or sequence composition between species because a variable region may include a DNA sequence that is not conversed between species. That is, variable regions between cotton species include one or more sequence or length polymorphisms, which are not conserved between species. The variation (i.e. polymorphism) may be, for example, a difference in sequence length within a genetic region, or single nucleotide changes within a specific genetic region. Variable regions (i.e. non-conserved regions) may exist between cotton species in nuclear DNA, mitochondrial DNA or chloroplast DNA of the cotton species. Non-variable regions have identical DNA sequences between species because the non-variable regions are conserved between species. Thus, the variable regions may be utilized as an endogenous marker to distinguish between cotton species.

Exemplary embodiments of the present invention relate to methods for assessing proportions of one or more cotton species included in an article including cotton. The method includes providing a sample including cotton fibers from the article including cotton. One or more cotton species included in the sample may be identified, and proportions of the one or more cotton species may be quantitatively assessed. The sample including cotton fibers may be obtained from a textile article, such as clothing or fabric. The textile article may include raw cotton fibers of one or more species of cotton.

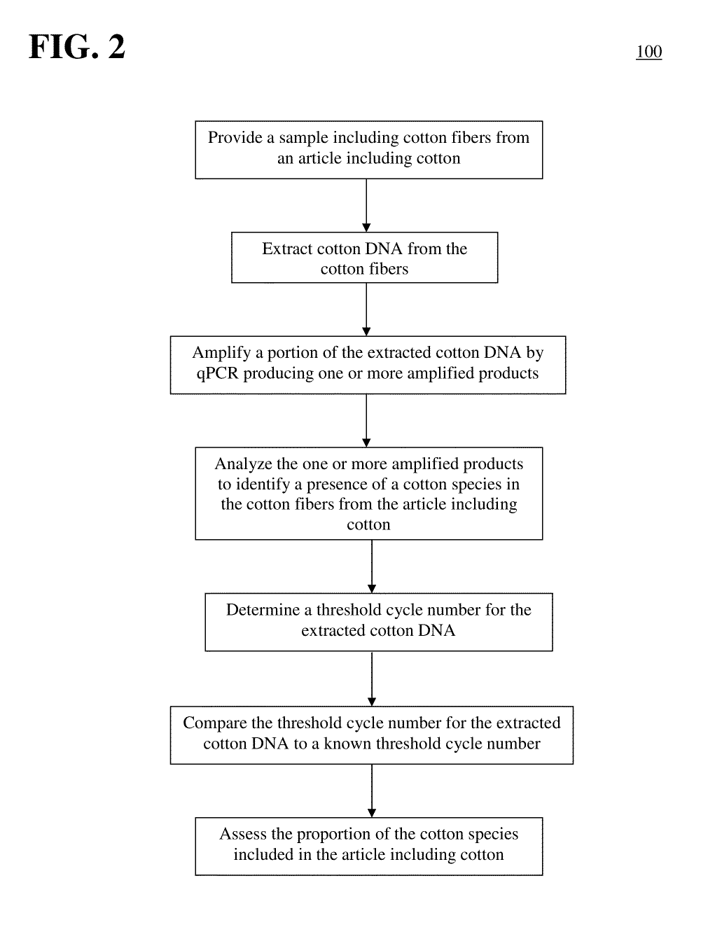

FIG. 2 is a flow chart illustrating a method of assessing a proportion of one or more cotton species included in an article including cotton according to an exemplary embodiment of the present invention. Referring to FIG. 2, method 100 (illustrated in FIG. 2) may include providing a sample including cotton fibers from an article including cotton. DNA may be extracted from the cotton fibers to produce extracted cotton DNA. A portion of the extracted cotton DNA may be amplified by qPCR and one or more amplified products may be produced. The one or more amplified products may be analyzed to identify a presence of one or more cotton species in the cotton fibers from the article including cotton. A threshold cycle number may be determined for the extracted cotton DNA. The threshold cycle number for the extracted cotton DNA may be compared to a known threshold cycle number. A proportion of one or more cotton species included in the article including cotton may be assessed.

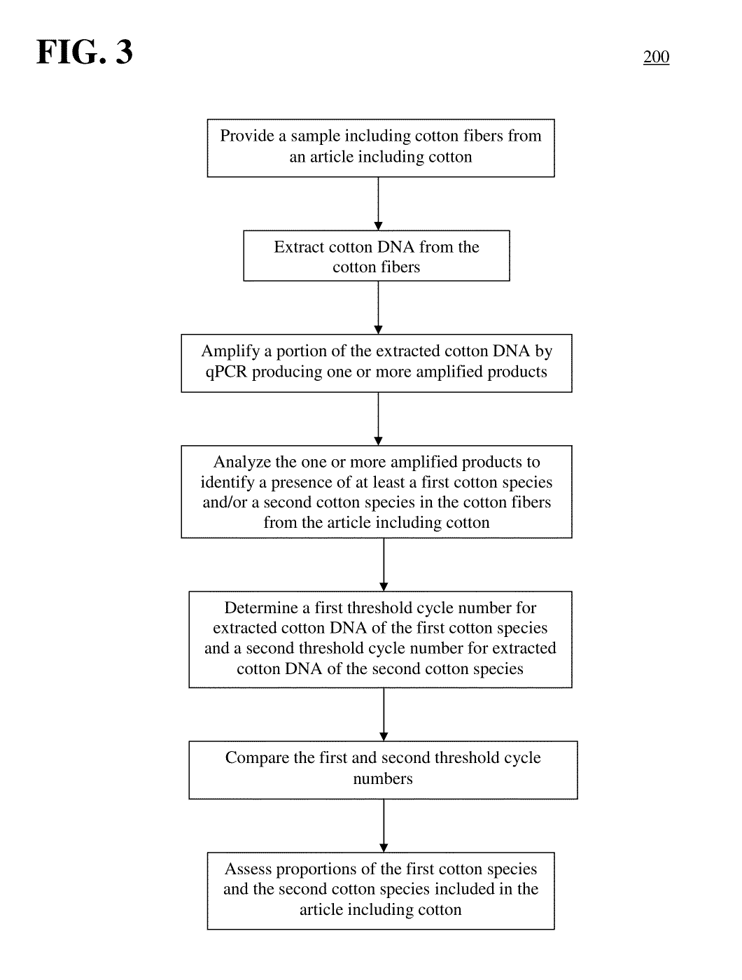

FIG. 3 is a flow chart illustrating a method of assessing a proportion of one or more cotton species included in an article including cotton according to an exemplary embodiment of the present invention. Referring to FIG. 3, method 200 (illustrated in FIG. 3) may include providing a sample including cotton fibers from an article including cotton. DNA may be extracted from the cotton fibers to produce extracted cotton DNA. A portion of the extracted cotton DNA may be amplified by qPCR and one or more amplified products may be produced. The one or more amplified products may be analyzed to identify a presence of at least a first cotton species and/or a second cotton species in the cotton fibers from the article including cotton. A first threshold cycle number for extracted cotton DNA of the first cotton species may be determined. A second threshold cycle number for extracted cotton DNA of the second cotton species may be determined. The first and second threshold cycle numbers may be compared to each other and proportions of the first cotton species and the second cotton species included in the article including cotton may be assessed.

### Cotton Fiber Sampling

According to exemplary embodiments of the present invention, the sample including cotton fibers is collected from the article including cotton. The collected cotton fibers may include mature cotton fibers. For example and without limitation, the sample including cotton fibers is obtained from raw cotton, which may be stored in cotton bales, or the sample including cotton fibers is obtained from an article such as a textile article.

The sample can be any suitable sample, such as for instance a solid sample, a scraping, a powder, a liquid, a mist, or a swab including cotton fibers. The cotton fiber sample can be collected in a collection vessel, such as a plastic or glass test tube, an eppendorf tube, a screw-cap tube, a microcap tube, a well of an assay plate or a microfluidic chamber, reservoir or other suitable container.

The sample including cotton fibers may be collected by scraping, cutting or dissolving a portion of the article to obtain cotton fibers for analysis. The collecting of the sample is carried out, for example, by cutting the article to remove cotton fibers from the article. According to exemplary embodiments of the present invention, the sample may be collected by tweezing, scraping, or abrading the article with appropriate sampling tools configured to remove a sufficient amount of cotton fibers or cotton lint for analysis. The sample including cotton fibers may be collected by using a collection kit. The sample collection kit according to an exemplary embodiment of the present invention includes a sample collection unit configured to collect a sample. For example and without limitation the sample including cotton fibers is a 2 cm by 2 cm square swatch removed from a textile article by cutting off a piece of the textile article. The swatch is transferred to a sample container, such as an eppendorf tube, for cotton DNA extraction from the collected cotton fibers.

Collecting the sample including cotton fibers may occur at any point along the supply or commerce chain where there is concern about or risk of introduction of counterfeit articles.

### Cotton DNA Extraction

According to exemplary embodiments of the present invention, DNA may be extracted from the cotton fibers to provide extracted cotton DNA. The extracted cotton DNA may include nuclear DNA, mitochondrial DNA and/or chloroplast DNA. For example and without limitation, cotton DNA is extracted from mature cotton fibers and the extracted cotton DNA includes chloroplast DNA.

Extraction of cotton DNA may include extraction, isolation and purification of the cotton DNA. A variety of nucleic acid extraction solutions have been developed for extracting DNA from a sample of interest. See, for example, Sambrook et al. (Eds.) Molecular Cloning, Cold Spring Harbor Press, 1989; and Green. Michael R., and Joseph Sambrook. Molecular cloning: a laboratory manual. New York: Cold Spring Harbor Laboratory Press, 2012. Many such methods include, for example, a detergent-mediated step, a proteinase treatment step, a phenol and/or chloroform extraction step, and/or an alcohol precipitation step. Some nucleic acid extraction solutions may include an ethylene glycol-type reagent or an ethylene glycol derivative to increase the efficiency of nucleic acid extraction while other methods only use grinding and/or boiling the sample in water. Other methods, including solvent-based systems and sonication, may also be utilized in conjunction with other extraction methods.

About 5 mg to about 30 mg of cotton fibers or cotton lint, and in certain instances between about 10 mg to about 15 mg of cotton fibers may be used for cotton DNA to be extracted from the cotton fibers for analysis. DNA extraction protocols may be derived from standard molecular biology DNA extraction procedures, which can be easily accomplished by persons skilled in the art.

While extracting DNA from cotton seeds and cotton leaves from various cotton species may be common for cotton genomics research, successful extraction of DNA from mature cotton fibers was not known before the present invention. The use of mature cotton fibers as a DNA source in the methods according to exemplary embodiments of the present invention permits identification of and distinguishing between at least two different cotton species and quantitatively assessing proportions of cotton species included in a sample including cotton fibers from an article such as a textile article. The methods according to the present invention allow for quantitative genetic analysis of one or more cotton species included in an article including cotton. The present invention provides methods for definitive identification of textiles including G. barbadense and/or G. hirsutum cotton, and also provides methods for quantitative percentage determination of each cotton species included in an article including cotton.

### Analysis and Cloning of Eukaryotic Genomic DNA

1. Depending on the type of sample, carry out one of the following procedures as step 1.

### Protocol I

Drop freshly excised tissue into liquid nitrogen in the stainless-steel cup of a Waring Blendor. Blend at top speed until the tissue is ground to a powder. Allow the liquid nitrogen to evaporate, and add the powdered tissue little by little to approximately 10 volumes of extraction buffer (10 mM Tris.Cl (pH 8.0), 0.1 mM EDTA (pH 8.0), 20 μg/ml pancreatic RNAase, 0.5% SDS) in a beaker. Allow the powder to spread over the surface of the extraction buffer, and then shake the beaker to submerge the material. When all of the material is in solution, transfer the solution to a 50-ml centrifuge tube, incubate for 1 hour at 37° C., and then proceed to step 2.

2. Add proteinase K to a final concentration of 100 μg/ml. Using a glass gently mix the enzyme into the viscous solution. Proteinase K is stored as a stock solution at a concentration of 20 mg/ml in H₂O.
3. Place the suspension of lysed cells in a water bath for 3 hours at 50° C. Swirl the viscous solution periodically.
4. Cool the solution to room temperature, and, if necessary, pour the solution into a centrifuge tube. Add an equal volume of phenol equilibrated with 0.5 M Tris.Cl (pH 8.0) and gently mix the two phases by slowly turning the tube end over end for 10 minutes. If the two phases have not formed an emulsion at this stage, place the tube on a roller apparatus for 1 hour. Separate the two phases by centrifugation at 5000g for 15 minutes at room temperature. It is essential that the pH of the phenol be approximately 8.0 to prevent DNA from becoming trapped at the interface between the organic and aqueous phases.
5. With a wide-bore pipette (0.3 cm-diameter orifice), transfer the viscous aqueous phase to a clean centrifuge tube and repeat the extraction with phenol twice. When transferring the aqueous phase, it is essential to draw the DNA into the pipette very slowly to avoid disturbing the material at the interface. If the DNA solution is so viscous that it cannot easily be drawn into a wide-bore pipette, use a long pipette attached to a water-suction vacuum pump to remove the organic phase. Make sure that the phenol is collected into traps and does not enter the water line. With the vacuum line closed, slowly lower the pipette to the bottom of the organic phase. Wait until the viscous thread of aqueous material detaches from the pipette, and then carefully open the vacuum line and gently withdraw all of the organic phase. Close the vacuum line and quickly withdraw the pipette through the aqueous phase. Immediately open the vacuum line to transfer the residual phenol into the trap. Centrifuge the DNA solution at 5000g for 20 minutes at room temperature. Protein and clots of DNA sediment to the bottom of the tube. Pour the DNA solution into a 50-ml centrifuge tube, leaving behind the protein and clots of DNA.
6. To isolate very-high-molecular-weight DNA (˜200 kb): After the third extraction with phenol, dialyze the pooled aqueous phases at 4° C. four times against 4 liters of a solution of 50 mM Tris.Cl (pH 8.0) 10 mM EDTA (pH 8.0) until the OD₂₇₀ of the dialysate is less than 0.05. Allow room in the dialysis bag for the volume of the sample to increase 1.5 to 2.0-fold. Continue to step 7.

To isolate DNA whose size is 100-150 kb: After the third extraction with phenol, transfer the pooled aqueous phases to a fresh centrifuge tube and add 0.2 volume of 10 M ammonium acetate. Add 2 volumes of ethanol at room temperature and swirl the tube until the solution is thoroughly mixed. The DNA will immediately form a precipitate that can usually be removed from the ethanolic solution with a pasteur pipette whose end has been sealed and shaped into a U. Most of the contaminating oligo-nucleotides are left behind. If the DNA precipitate becomes fragmented collect it by centrifugation at 5000g for 5 minutes at room temperature in a swinging-bucket rotor. Wash the DNA precipitate twice with 70% ethanol, and collect the DNA by centrifugation as described above. Remove as much as possible of the 70% ethanol, and store the pellet in an open tube at room temperature until the last visible traces of ethanol have evaporated. Do not allow the pellet of DNA to dry completely; otherwise, it will be very difficult to dissolve. Add 1 ml of TE (pH 8.0) for each ˜5×10⁶ cells. Place the tube on a rocking platform and gently rock the solution until the DNA has completely dissolved. This usually takes 12-24 hours.

7. Measure the absorbance of the DNA at 260 nm and 280 nm. The ratio of A₂₆₀ to A₂₈₀ should be greater than 1.75. A lower ratio is an indication that significant amounts of protein remain in the preparation. In this case, add SDS to a concentration of 0.5% and then repeat steps 2-7.
8. Calculate the concentration of the DNA (a solution with an OD₂₆₀ of 1 contains approximately 50 μg of DNA per milliliter), and analyze an aliquot by pulsed-field gel electrophoresis or by electrophoresis through a 0.3% agarose gel poured on a 1% agarose support. The DNA should be larger than 100 kb in size and should migrate more slowly than linear dimeric molecules of intact bacteriophage λ DNA. Store the DNA at 4° C.

### Protocol II

This method, which is adapted from Bowtell (1987), is used to prepare DNA simultaneously from many different samples of cells or tissues.

1. Prepare cell suspensions (or frozen cell powders) as described in step 1 of protocol I.
2. Transfer the cell suspensions to centrifuge tubes, and add 7.5 volumes of lysis solution consisting of 6 M guanidine HCl (Mᵣ=95.6), 0.1 M sodium acetate (pH 5.5).

If the DNA is to be extracted from tissues, add the frozen cell powders to approximately 7.5 volumes of lysis solution in beakers. Allow the powders to spread over the surface of the lysis solution, and then shake the beakers to submerge the material. When all the material is in solution, transfer the solution to centrifuge tubes.

3. Close the tops of the tubes and incubate for 1 hour at room temperature on a rocking platform.
4. Dispense 18 ml of ethanol at room temperature into each of a series of disposable 50-ml polypropylene centrifuge tubes. Using wide-bore pipettes, carefully layer the cell suspensions under the ethanol.
5. Recover the DNA from each tube by slowly stirring the interface between the cell lysate and the ethanol with a pasteur pipette whose end has been sealed and bent into a U shape. The DNA will adhere to the Pasteur pipette, forming a gelatinous mass. Continue stirring until the ethanol and the aqueous phase are thoroughly mixed.
6. Transfer each pasteur pipette, with its attached DNA, to a separate polypropylene tube containing 5 ml of ethanol at room temperature. Leave the DNA submerged in the ethanol until all of the samples have been processed.
7. Remove each pipette, with its attached DNA, and allow as much ethanol as possible to drain away. By this stage, the DNA should have shrunk into a tightly packed, dehydrated mass, and it is often possible to remove most of the free ethanol by capillary action by touching the U-shaped end of the pipette to a stack of Kimwipes. Before all of the ethanol has evaporated from the DNA, transfer the pipette into a fresh polypropylene tube containing 5 ml of ethanol at room temperature.
8. When all of the samples have been processed, remove each pipette with its attached DNA, and remove as much ethanol as possible as described in step 7 above. Do not allow the pellet of DNA to dry completely—otherwise, it will be very difficult to dissolve.
9. Transfer each pipette to a fresh polypropylene tube containing 1 ml of TE (pH 8.0). Allow the DNAs to rehydrate by storing the tubes overnight at 4° C.
10. By the next morning, the DNAs will have become highly gelatinous but will still be attached to their pipettes. Using fresh, sealed pasteur pipettes as scrapers, gently free the pellets of DNA from their pipettes. Discard the pipettes, leaving the DNAs floating in the TE. Close the tops of the tubes and incubate the DNAs at 4° C. on a rocking platform until they are completely dissolved. This often takes 24-48 hours.
11. Analyze an aliquot by pulsed-field gel electrophoresis or by electrophoresis through a 0.3% agarose gel poured on a 1% agarose support. The DNA should be ˜80 kb in size and should migrate more slowly than monomers. Store the DNA at 4° C.

### NOTES

i. DNA made by this procedure is always contaminated with a small amount of RNA. It is therefore necessary to estimate the concentration of DNA in the final preparation either by fluorimetry or by gel electrophoresis and staining with ethidium bromide. If desired, the amount of contaminating RNA can be minimized by transferring the rehydrated pellet of DNA (step 10) to a fresh polypropylene tube containing 1 ml of TE (pH 8.0) before scraping it from the pasteur pipette. This is a hazardous procedure, since there is a risk that the DNA will slide off the pipette during transfer.
Partial Digestion of Eukaryotic DNA with Restriction Enzymes

The only method by which DNA can be fragmented in truly random fashion irrespective of its base composition and sequence, is mechanical shearing. However, DNA prepared in this way requires several additional enzymatic manipulations (repair of termini, methylation, ligation to linkers, digestion of linkers) to generate cohesive termini compatible with those of the vectors used to generate genomic DNA libraries (Maniatis et al. 1978). One hand, partial digestion with restriction enzymes that recognize frequently occurring tetranucleotide sequences within eukaryotic DNA yields a population of fragments that is close to random and yet can be cloned directly.

Fragments of eukaryotic DNA suitable for the construction of genomic DNA libraries are prepared as follows: Carry out pilot experiments to establish conditions for partial digestion of eukaryotic DNA. Guided by the results of the pilot experiments, digest a large amount of eukaryotic DNA and purify fragments of the desired size by density gradient centrifugation.

### Pilot Experiments

1. Dilute 30 μg of high-molecular-weight eukaryotic DNA (>200 kb; see Protocol I) to 900 μl with 10 mM Tris.Cl (pH 8.0) and add 100 μl of the appropriate 10× restriction enzyme buffer.

If the concentration of the high-molecular-weight DNA is low, increase the volume of the pilot reactions and concentrate the DNA after digestion by precipitation with ethanol. Each pilot reaction should contain at least 1 μg of DNA to allow the heterogeneous products of digestion to be detected by staining with ethidium bromide. Handle the eukaryotic DNA carefully by using either pipette tips that have been cut off with a sterile razor blade to enlarge the orifice or disposable wide-bore glass capillaries. Make sure that the DNA is dispersed homogeneously throughout the buffer used for digestion. The chief problem encountered during digestion of high-molecular-weight DNA is unevenness of digestion caused by variations in the local concentration of DNA. Clumps of DNA are relatively inaccessible to restriction enzymes and can be digested only from the outside. Unless the DNA is evenly dispersed, the rate of digestion cannot be predicted or controlled. To ensure homogeneous dispersion of the DNA:

a. Allow the DNA to stand at 4° C. for several hours after dilution and addition of 10× restriction enzyme buffer.
b. Gently stir the DNA solution from time to time using a sealed glass capillary.
c. After addition of the restriction enzyme, gently stir the solution for 2-3 minutes at 4° C. before warming the reaction to the appropriate temperature.
d. After digestion for 15-30 minutes, add a second aliquot of restriction enzyme and stir the reaction as described above.
2. Carry out test digestions on aliquots of the batch of diluted DNA that will be used to prepare fragments for cloning. The amount of enzyme necessary will vary for each batch of enzyme and preparation of DNA.
a. Using a wide-bore glass capillary or a cut-off disposable plastic pipette tip, transfer 60 μl of the DNA solution to a microfuge tube (tube 1). Transfer 30 μl of the DNA solution to each of nine additional labeled microfuge tubes. Stand the tubes on ice.
b. Add 2 units of the appropriate restriction enzyme to the first tube. Use a sealed glass capillary to mix the restriction enzyme with the DNA. Do not allow the temperature of the reaction to rise above 4° C. Using a fresh pipette tip, transfer 30 μl of the reaction to the next tube in the series. Mix as before, and continue transferring the reaction to successive tubes. Do not add anything to the tenth tube (no enzyme control), but discard 30 μl from the ninth tube. Incubate the reactions for 1 hour at 37° C.
c. At the end of the digestion, heat the reactions to 70° C. for 15 minutes to inactivate the restriction enzyme. After cooling the reactions to room temperature, add the appropriate amount of gel-loading buffer (0.25% bromophenol blue, 0.25% xylene cyanol FF, 30% glycerol in water). Mix the solutions gently, using a sealed glass capillary. Use a cut-off, disposable plastic pipette tip or a disposable wide-bore glass capillary to transfer the solutions to the wells of a 0.3% agarose gel poured on a 1% agarose support. Compare the size of the digested eukaryotic DNA with that of oligomers of bacteriophage λ DNA and plasmids (see notes to step 1).
Large scale Preparation of Partially Digested DNA
1. After conditions have been established in pilot experiments (see above), digest 100 μg of high-molecular-weight DNA with the appropriate amount of restriction enzyme for the appropriate time. To ensure that the conditions for the large-scale digestion are as identical as possible to those used in the pilot experiment, we prefer to set up replicas of the successful pilot reaction rather than a single large-scale reaction. In addition, if sufficient eukaryotic DNA is available, we recommend using three different concentrations of restriction enzyme that straddle the optimal concentration determined in the pilot experiment. At the end of the digestion, analyze an aliquot of the DNA in each digestion by gel electro-phoresis to make sure that the digestion has worked according to prediction. Until the results are available, store the remainder of the sample at 0° C.
2. Gently extract the digested DNA twice with phenol:chloroform. Precipitate the DNA with 2 volumes of ethanol at 0° C. and redissolve it in 200 μl of TE (pH 8.0).
3. Prepare a 10-40% continuous sucrose density gradient in a Beckman SW40 polyallomer tube (or its equivalent). The sucrose solutions are made in a buffer containing 10 mM Tris.Cl (pH 8.0), 10 mM NaCl, 1 mM EDTA (pH 8.0). Heat the DNA sample for 10 minutes at 68° C., cool it to 20° C., and load it onto the gradient. Centrifuge at 22,000 rpm for 22 hours at 20° C. in a Beckman SW40 rotor (or its equivalent).
4. Using a 19-gauge needle, puncture the bottom of the tube and collect 350-μl fractions.
5. Mix 10 μl of every other fraction with 10 μl of water and 5 μl of gel-loading buffer I (0.25% bromophenol blue, 0.25% xylene cyanol FF, 40% (w/v) sucrose in water). Analyze the size of the DNA in each fraction by electrophoresis through a 0.5% agarose gel, using oligomers of plasmid DNA as markers. Be sure to adjust the sucrose and salt concentrations of the markers to correspond to those of the samples.
6. Following electrophoresis, pool the gradient fractions containing DNA fragments of the desired size. Dialyze the pooled fractions against 2 liters of TE (pH 8.0) at 4° C. for 12-16 hours, with a change of buffer after 4-6 hours. Leave space in the dialysis bag for the sample to expand two- to threefold. Alternatively, if the volume of the pooled sample is sufficiently small, the DNA can be precipitated with ethanol without prior dialysis after first diluting the sample with TE (pH 8.0) so that the concentration of sucrose is reduced to below 10%.
7. Extract the dialyzed DNA several times with an equal volume of 2-butanol until the volume is reduced to about 1 ml. Add 10 M ammonium acetate to a final concentration of 2 M and precipitate the DNA with 2 volumes of ethanol at room temperature.
8. Dissolve the DNA in TE (pH 8.0) at a concentration of 300-500 μg/ml. Analyze an aliquot of the DNA (0.5 μg) by electrophoresis through a 0.5% agarose gel to check that the size distribution of the digestion products is correct.

### Large Scale Preparation of Partially Digested DNA

1. After conditions have been established in pilot experiments (see above), digest 100 pg of high-molecular-weight DNA with the appropriate amount of restriction enzyme for the appropriate time. To ensure that the conditions for the large-scale digestion are as identical as possible to those used in the pilot experiment, we prefer to set up replicas of the successful pilot reaction rather than a single large-scale reaction. In addition, if sufficient eukaryotic DNA is available, we recommend using three different concentrations of restriction enzyme that straddle the optimal concentration determined in the pilot experiment. At the end of the digestion, analyze an aliquot of the DNA in each digestion by gel electrophoresis to make sure that the digestion has worked according to prediction. Until the results are available, store the remainder of the sample at 0° C.
2. Gently extract the digested DNA twice with phenol xhloroform. Precipitate the DNA with 2 volumes of ethanol at 0° C. and redissolve it in 200 μl of TE (pH 8.0).
3. Prepare a 10-40% continuous sucrose density gradient in a Beckman SW40 polyallomer tube (or its equivalent). The sucrose solutions are made in a buffer containing 10 mM Tris.Cl (pH 8.0), 10 mM NaCl, 1 mM EDTA (pH 8.0). Heat the DNA sample for 10 minutes at 68° C., cool it to 20° C., and load it onto the gradient. Centrifuge at 22,000 rpm for 22 hours at 20° C. in a Beckman SW40 rotor (or its equivalent).
4. Using a 19-gauge needle, puncture the bottom of the tube and collect 350-pl fractions.
5. Mix 10 μl of every other fraction with 10 μl of water and 5 μl of gel-loading buffer I (0.25% bromophenol blue, 0.25% xylene cyanol FF, 40% (w/v) sucrose in water). Analyze the size of the DNA in each fraction by electrophoresis through a 0.5% agarose gel, using oligomers of plasmid DNA as markers. Be sure to adjust the sucrose and salt concentrations of the markers to correspond to those of the samples.
6. Following electrophoresis, pool the gradient fractions containing DNA fragments of the desired size (e.g., 35-45 kb for construction of libraries in cosmids; 20-25 kb for construction of libraries in bacteriophage λ vectors such as EMBL3 and 4). Dialyze the pooled fractions against 2 liters of TE (pH 8.0) at 4° C. for 12-16 hours, with a change of buffer after 4-6 hours. Leave space in the dialysis bag for the sample to expand two to threefold. Alternatively, if the volume of the pooled sample is sufficiently small, the DNA can be precipitated with ethanol without prior dialysis after first diluting the sample with TE (pH 8.0) so that the concentration of sucrose is reduced to below 10%.

### Exemplary DNA Extraction Protocol III

10-15 mg of a cotton fiber sample is transferred to a 1.5 ml eppendorf tube for DNA extraction. 200 μL of Extraction Buffer (e.g., Part #E7526, Sigma-Aldrich, St. Louis, Mo.—discussed in more detail below) is added to the eppendorf tube containing the sample. The Extraction Buffer and the sample are incubated at 95° C. for 30 minutes. After 30 minutes 200 μL of Dilution Buffer (e.g., Part #D5688, Sigma-Aldrich—discussed in more detail below) are added to the Extraction Buffer. The Dilution Buffer is added to stop the reaction between the Extraction Buffer and the cotton fiber sample and the eppendorf tube is then vortexed until the contents were well mixed.

The eppendorf tube is then transferred to a DNA IQ spin basket (e.g., Part # V1221, Promega Corporation, Madison, Wis.). The eppendorf tube is then centrifuged at 13,000 RPM for about 30 second and the cotton DNA included in the resulting sample may then be used as a PCR template for qPCR.

The use of the Extraction Buffer and the Dilution buffer, as well as exemplary DNA extraction protocols are discussed in more detail, for example, in Flores, Gilberto E., Jessica B. Henley, and Noah Fierer. A direct PCR approach to accelerate analyses of human-associated microbial communities. PloS one 7.9 (2012): e44563.

### DNA Amplification

Cotton DNA is extracted from the cotton fibers to provide extracted cotton DNA. The extracted cotton DNA is amplified and one or more amplified products (e.g., amplicons) are generated. Cotton DNA amplification may include a polymerase chain reaction (PCR). For example and without limitation, the cotton DNA is amplified by qPCR. The extracted cotton DNA may include chloroplast DNA. According to an exemplary embodiment of the present invention, the amplified portion of the extracted cotton DNA is amplified by using chloroplast DNA as a template.

Quantitative Real-Time PCR (qPCR) Amplification

Quantitative Real-Time PCR (qPCR) may also be referred to as RT-PCR or RT-qPCR and these terms may be used interchangeably throughout this application. qPCR may be run singleplex or multiplex. As discussed below in more detail, fluorescence signals increase during qPCR thermal cycling and amplification, and a threshold cycle number may be determined based on an amplification curve for each extracted DNA template. The threshold cycle number may be determined automatically by an automated qPCR instrument (e.g. ABI 7900HT, Life Technologies, Grand Island, N.Y.). The threshold cycle number is directly proportional to the amount of DNA template extracted from the cotton fibers of each species of cotton included in the article including cotton. Based on the threshold cycle number for the extracted DNA template of each cotton species included in the article including cotton, the proportion or percentage of each species of cotton included in a particular article is determined. If there is only one amplification curve detected, then the sample includes only one species of cotton and is therefore 100% pure.

### DNA Primers

Portions of the extracted cotton DNA may be amplified using at least one set of specific primers and one or more amplified products may be produced. According to an exemplary embodiment of the present invention, the specific primers are complementary to non-variable regions of the one or more cotton species. For example and without limitation, the specific primers are complementary to non-variable regions that are conserved between ELS and non-ELS cotton species (see, e.g., FIG. 1). According to an exemplary embodiment of the present invention, the conserved non-variable regions are in the chloroplast DNA of ELS and non-ELS cotton species. For example and without limitation, the ELS species is G. barbadense and the non-ELS species is G. hirsutum. Thus, a single set of specific primers can be used to simultaneously amplify ELS and non-ELS cotton species (e.g., G. barbadense and G. hirsutum).

According to an exemplary embodiment of the present invention, the following set of primers may be used to simultaneously amplify portions of the extracted cotton DNA of ELS and non-ELS cotton species (e.g., G. barbadense and G. hirsutum). The following set of primers was synthesized by Life Technologies.

Forward primer sequence:
5′-AAT CCC AGG GAA ATA AAG AAA AGT GTA-3′;
Reverse primer sequence:
5′-TTA CAA CCC GGC TTC GAA TCT A-3′.

Referring to FIG. 1, the forward and reverse primers are complementary to non-variable regions of ELS and non-ELS cotton species in relatively close proximity and on opposite sides of a variable region of the ELS and non-ELS cotton species.

### Cotton DNA Analysis

Cotton DNA extracted from cotton fibers may be analyzed to assess any desired information related to the extracted cotton DNA without limitation. For example, cotton DNA extracted from the cotton fibers may be analyzed to detect the presence of any nucleic acid sequence in the extracted cotton DNA such as a nucleic acid sequence including a single nucleotide polymorphism or a sequence length polymorphism.

Cotton DNA may be amplified by Multiple Annealing and Looping Based Amplification Cycles (MALBAC). MALBAC may be used to amplify substantially a whole genome. MALBAC operates in quasi-linear fashion and may be used for single cell, whole genome amplification. In MALBAC, amplicons may have complementary ends. The complementary ends may form loops, which may prevent exponential amplicon copying, thus preventing amplification bias. MALBAC is discussed in more detail in Zong, Chenghang, et al. “Genome-wide detection of single-nucleotide and copy-number variations of a single human cell” Science 338.6114 (2012): 1622-1626.

Molecular beacons systems may be used with real time PCR for quantitatively detecting DNA in a sample. For example, the commercially available Roche Light Cycler™ (Roche Diagnostics Corporation, Indianapolis, Ind.), or other such instruments may be used for this purpose. A molecular beacon probe may be visible under daylight or conventional lighting and/or may be fluorescent. Multicolor molecular beacons are discussed in Tyagi, Sanjay, Diana P. Bratu, and Fred Russell Kramer. “Multicolor molecular beacons for allele discrimination” Nature biotechnology 16.1 (1998): 49-53. Fluorescent molecular beacons are discussed in Tyagi, Sanjay, and Fred Russell Kramer. “Molecular beacons: probes that fluoresce upon hybridization” Nature biotechnology 14.3 (1996): 303-308.

Generally, PCR employs reiterative thermal cycling to amplify DNA. However, DNA can be amplified without thermal cycling (e.g., by isothermal amplification). For example, DNA may be isothermally amplified by Loop-mediated isothermal amplification (LAMP), Helicase-dependent amplification (HDA), Nicking enzyme amplification reaction (NEAR), Strand displacement amplification (SDA), Recombinase Polymerase Amplification (RPA), or thermophilic helicase dependent amplification (tHDA). Isothermal amplification techniques are discussed in more detail in Oriero, E. C., et al. “Comparison of two isothermal amplification methods: Thermophilic helicase dependent amplification (tHDA) and loop mediated isothermal amplification (LAMP) for detection of Plasmodium falciparum” International Journal of Infectious Diseases 21 (2014): 381; and Li, Ying, et al. “Detection and Species Identification of Malaria Parasites by Isothermal tHDA Amplification Directly from Human Blood without Sample Preparation” The Journal of Molecular Diagnostics 15.5 (2013): 634-641.

Cotton DNA may be amplified by Strand Displacement Amplification (SDA). SDA is a method of DNA amplification that is non-sequence-specific. Random hexamer primers are annealed to a DNA template strand and DNA synthesis is performed by a high fidelity DNA polymerase. SDS is discussed in more detail in U.S. Pat. No. 5,455,166; U.S. Pat. No. 5,712,124; Asiello, Peter J., and Antje J. Baeumner. “Miniaturized isothermal nucleic acid amplification, a review” Lab on a Chip 11.8 (2011): 1420-1430; and Walker, G. Terrance, et al. “Strand displacement amplification—an isothermal, in vitro DNA amplification technique” Nucleic Acids Research 20.7 (1992): 1691-1696.

Cotton DNA may be amplified by Loop-mediated isothermal amplification (LAMP). LAMP may also be referred to as a single tube technique for amplifying DNA. In LAMP, all reagents are incubated in a single sample tube. LAMP employs a DNA polymerase with strand displacement properties, and a thermocycler need not be used. LAMP may include using a set of primers (e.g., 4 or 6 primers) targeting a set of regions (e.g., 6 or 8 regions) within a relatively small target DNA sequence. LAMP may employ a pair of inner and a pair of outer primers, plus two additional loop-primers, which may anneal at a loop structure in LAMP amplicons. This design enhances amplification sensitivity while reducing reaction time. LAMP is discussed in more detail in Oriero, E. C., et al. “Comparison of two isothermal amplification methods: Thermophilic helicase dependent amplification (tHDA) and loop mediated isothermal amplification (LAMP) for detection of Plasmodium falciparum” International Journal of Infectious Diseases 21 (2014): 381; and Notomi, Tsugunori, et al. “Loop-mediated isothermal amplification of DNA” Nucleic acids research 28.12 (2000): e63-e63.

Cotton DNA may be amplified by Nicking Enzyme Amplification Reaction (NEAR) amplification. NEAR amplification employs a strand-displacing DNA polymerase to synthesize DNA from a nick created in DNA by a nicking enzyme. NEAR produces many relatively short nucleic acids from a target sequence in a relatively short period of time. Alternating cycles of nicking and DNA extension may result in billion-fold amplification within 5-10 minutes. NEAR is discussed in more detail in Ménová, Petra, Veronika Raindlova, and Michal Hocek. “Scope and Limitations of the Nicking Enzyme Amplification Reaction for the Synthesis of Base-Modified Oligonucleotides and Primers for PCR” Bioconjugate Chemistry 24.6 (2013): 1081-1093.

Cotton DNA may be amplified by Recombinase Polymerase Amplification (RPA). RPA may also be referred to as a single tube technique for DNA amplification. RPA includes three enzymes—a recombinase, a single-stranded DNA-binding protein (SSB) and strand-displacing polymerase. The recombinase pairs oligonucleotide primers with homologous sequence in duplex DNA. The SSB binds to displaced strands of DNA and prevents displacement of the primers. The strand displacing polymerase starts DNA synthesis at a point where the primer is bound to a target DNA. A reverse transcriptase enzyme is added to an RPA reaction to detect RNA and/or DNA without producing cDNA. RPA is discussed in more detail in Lutz, Sascha, et al. “Microfluidic lab-on-a-foil for nucleic acid analysis based on isothermal recombinase polymerase amplification (RPA)” Lab on a Chip 10.7 (2010): 887-893.

Cotton DNA may be amplified by thermophilic helicase dependent amplification (tHDA). tHDA selectively amplifies a target DNA sequence. For example, tHDA may amplify a relatively short cotton DNA sequence of about 70 bp to 120 bp, defined by two primers. tHDA includes using a helicase to separate DNA (instead of heat), thus generating single stranded DNA templates for primer binding and extension by DNA polymerase. tHDA can amplify DNA from even a single copy of a DNA template. tHDA is discussed in more detail in Oriero, E. C., et al. “Comparison of two isothermal amplification methods: Thermophilic helicase dependent amplification (tHDA) and loop mediated isothermal amplification (LAMP) for detection of Plasmodium falciparum” International Journal of Infectious Diseases 21 (2014): 381; and Li, Ying, et al. “Detection and Species Identification of Malaria Parasites by Isothermal tHDA Amplification Directly from Human Blood without Sample Preparation” The Journal of Molecular Diagnostics 15.5 (2013): 634-641.

### Next-Generation Sequencing

Cotton DNA may be sequenced and/or detected by a next-generating sequencing (NGS) technology. NGS refers to a category of high-throughput sequencing technologies (e.g., massively parallel sequencing), which may identify the nucleic acid sequences of nuclear, mitochondrial and/or chloroplast DNA extracted from cotton fibers (e.g., mature cotton fibers). NGS technology may sequence relatively large nucleic acid sequences or an entire genome. In NGS, multiple relatively small nucleic acid sequences may be sequenced simultaneously from a DNA sample and a library of small segments (i.e., reads) may be built. The individual reads may then be reassembled to provide the sequence of a larger nucleic acid sequence or a complete nucleic acid sequence. For example and without limitation, 500,000 sequencing operations may be run in parallel. For instance, NGS may employ MALBAC followed by traditional PCR. NGS is discussed in more detail in Mardis, Elaine R. “The impact of next-generation sequencing technology on genetics” Trends in genetics 24.3 (2008): 133-141; and Metzker, Michael L. “Sequencing technologies—the next generation” Nature Reviews Genetics 11.1 (2009): 31-46.

Polony Sequencing is an example of NGS technology in which millions of immobilized DNA sequences are read in parallel. Polony sequencing is a multiplex sequencing technique in which a number of analytes are measured in a single run/cycle or a single assay. Polony sequencing has been shown to be extremely accurate with a low error rate. Polony Sequencing methods are discussed in more detail in Shendure, Jay, et al. “Advanced sequencing technologies: methods and goals” Nature Reviews Genetics 5.5 (2004): 335-344; and Shendure, Jay, and Hanlee Ji. “Next-generation DNA sequencing” Nature biotechnology 26.10 (2008): 1135-1145.

Massively Parallel Signature Sequencing (MPSS) is another example of NGS technology. MPSS can be utilized to both identify and quantify mRNA transcripts in a sample. MPSS identifies mRNA transcripts by generating 17-20 base pair signature sequences. MPSS methods are discussed in Brenner, Sydney, et al. “Gene expression analysis by massively parallel signature sequencing (MPSS) on microbead arrays” Nature biotechnology 18.6 (2000): 630-634.

Illumina Sequencing is an example of NGS technology in which DNA molecules and primers are immobilized on a slide. The immobilized DNA molecules may be amplified by a polymerase and DNA colonies (i.e., DNA clusters) are formed. Illumina Sequencing methods are discussed in more detail in Hanlee Ji. “Next-generation DNA sequencing” Nature biotechnology 26.10 (2008): 1135-1145; and Meyer, Matthias, and Martin Kircher. “Illumina sequencing library preparation for highly multiplexed target capture and sequencing” Cold Spring Harbor Protocols 2010.6 (2010): pdb-prot5448.

Pyrosequencing is an exemplary NGS technology in which luciferase is employed to detect individual nucleotides added to a nascent DNA. Pyrosequencing amplifies DNA contained in droplets of water in an oil solution. Each droplet of water may include, for instance, one DNA template attached to a primer-coated bead. Pyrosequencing methods are discussed in more detail in Vera, J. Cristobal, et al. “Rapid transcriptome characterization for a nonmodel organism using 454 pyrosequencing” Molecular ecology 17.7 (2008): 1636-1647; and Ronaghi, Mostafa. “Pyrosequencing sheds light on DNA sequencing” Genome research 11.1 (2001): 3-11.

Oligonucleotide Ligation and Detection (SOLiD Sequencing) is an example of NGS technology in which thousands of relatively small sequence reads (i.e., DNA fragments) are simultaneously generated. SOLiD sequencing may be referred to as a sequencing by ligation method. The sequence reads may be immobilized on a solid support for sequencing. SOLiD sequencing methods are discussed in more detail in Hanlee Ji. “Next-generation DNA sequencing” Nature biotechnology 26.10 (2008): 1135-1145; and Meyer, Matthias, and Ansorge, Wilhelm J. “Next-generation DNA sequencing techniques” New biotechnology 25.4 (2009): 195-203.

Ion Torrent Semiconductor Sequencing is another example of NGS technology in which hydrogen ions are released and detected during DNA polymerization. Ion Torrent Semiconductor Sequencing is an example of a sequence-by-synthesis method. A deoxyribonucleotide triphosphate (dNTP) may be provided into a microwell holding a template DNA strand. If the dNTP is complementary to a leading template nucleotide, the dNTP may be incorporated into the complementary DNA strand and a hydrogen ion will be released. Ion Torrent Semiconductor Sequencing methods are discussed in more detail in Quail, Michael A., et al. “A tale of three next generation sequencing platforms: comparison of Ion Torrent, Pacific Biosciences and Illumina MiSeq sequencers” BMC genomics 13.1 (2012): 341.

Heliscope Single Molecule Sequencing is an example of NGS technology that does not require PCR amplification. Heliscope Single Molecule Sequencing is an example of a direct-sequencing method in which DNA may be sheared, tailed with a poly-A tail and then hybridized to a surface of a flow cell. Relatively large numbers of molecules (e.g., billions of nucleotides) may be sequenced in parallel. Heliscope Single Molecule Sequencing methods are discussed in more detail in Pushkarev, Dmitry, Norma F. Neff, and Stephen R. Quake. “Single-molecule sequencing of an individual human genome” Nature biotechnology 27.9 (2009): 847-850.

DNA Nanoball Sequencing is an example of NGS technology, in which relatively small fragments of DNA are amplified using rolling circle replication to form DNA nanoballs. Amplified DNA sequences are ligated through the use of fluorescent probes as guides. DNA Nanoball Sequencing methods are discussed in more detail in Ansorge, Wilhelm J. “Next-generation DNA sequencing techniques” New biotechnology 25.4 (2009): 195-203, and Drmanac, Radoje, et al. “Human genome sequencing using unchained base reads on self-assembling DNA nanoarrays” Science 327.5961 (2010): 78-81.

Single Molecule Real Time (SMRT) Sequencing is another example of NGS technology, in which DNA may be synthesized in relatively small containers referred to as zero-mode wave-guides (ZMWs). Unmodified polymerases may be attached to bottoms of the ZMWs. The unmodified polymerases may be used to sequence the DNA along with fluorescently labeled nucleotides which are allowed to flow freely in the solution. Fluorescent labels may be released from each of the nucleotides as the nucleotides are incorporated into a DNA strand. SMRT sequencing is another example of a sequencing-by-synthesis method. SMRT Sequencing methods are discussed in more detail in Flusberg, Benjamin A., et al. “Direct detection of DNA methylation during single-molecule, real-time sequencing” Nature methods 7.6 (2010): 461-465.

### Identifying Cotton Species

According to exemplary embodiments of the present invention, the one or more amplified products are analyzed to identify a presence of at least one cotton species in the cotton fibers extracted from the article including cotton.

The article including cotton may include at least one cotton species and may include a blend of two or more species of cotton. For example and without limitation, the article including cotton may include a blend of G. barbadense and G. hirsutum cotton. The article including cotton may also include three or more species of cotton, such as a blend of G. barbadense, G. hirsutum, G. arboretum and/or G. herbaceum cotton.

### Hybridization Probes

One or more hybridization probes may be used to identify the presence of one or more cotton species included in the article including cotton. Each of the one or more hybridization probes is complementary to a variable region between ELS and non-ELS cotton species (see, e.g., FIG. 1). According to an exemplary embodiment of the present invention, the non-conserved variable region may be in the chloroplast DNA of ELS and non-ELS cotton species. For example and without limitation, the ELS species may be G. barbadense and the non-ELS species may be G. hirsutum. Thus, a first hybridization probe can identify an ELS cotton species (e.g., G. barbadense) and a second hybridization probe can identify a non-ELS cotton species (e.g., G. hirsutum).

Each of the hybridization probes may include a detectable marker. According to exemplary embodiments of the present invention, the detectable markers may be a fluorescent marker. Each of the fluorescent markers emits a distinct wavelength of light or light having a wavelength within a distinct range. The fluorescent markers are used to distinguish a first cotton species from a second cotton species. The hybridization probe including the fluorescent marker may include a fluorescently labeled nucleotide probe (e.g., a fluorescent reporter dye) at the 5′ end of the hybridization probe and a quencher at the 3′ end of the hybridization probe. The 5′ fluorescent reporter dye and the 3′ quencher may be selected as a fluorescent reporter dye-quencher pair. One example of a fluorescent reporter dye-quencher pair is fluorescein dye (e.g., fluorescein amidite (FAM), which is commercially available as 6-FAM), which emits green light, and Black Hole Quencher 1 dye. When the fluorescent reporter dye and the quencher are in close proximity, the fluorescence of the reporter is quenched due to its proximity to the quencher. The hybridization probe including an intact reporter-quencher pair anneals to a complementary DNA sequence of the variable region without emitting detectable light. During extension, the fluorescently labeled nucleotide probe is released from the hybridization probe and separated from its corresponding quencher and the fluorescent signal of the fluorescent dye becomes detectable. As the number of amplified PCR products increases due to repeated cycles of PCR, the intensity of the fluorescent signal detected as a result of an increased number of separated fluorescently labeled nucleotide probes increases. The fluorescent signal can be detected by a qPCR instrument (e.g. ABI 7900HT).

According to an exemplary embodiment of the present invention, the following ELS probe sequence may be labeled with a FAM fluorophore:

5′-6-FAM-ATG ATT TCA TTC AAG CCA TTT-MGBNFQ-3′
(Part# 4316033, Life Technologies);

According to an exemplary embodiment of the present invention, the following Upland probe sequence may be labeled with a VIC fluorophore:

5′-VIC-TCT TAT GAT TTC ATT CAT TTT C-MGBNFQ-3′
(Part# 4316033, Life Technologies).

Fluorescent reporter dyes for the ELS probe and the Upland probe with readily distinguishable emission spectra may be selected. Quenchers corresponding to the particular fluorescent reporter dyes of the ELS probe and the Upland probe, respectively, may be selected to have absorbance spectra which correspond with the emission spectra of their corresponding Fluorescent reporter dyes. According to exemplary embodiments of the present invention, the fluorescent reporter dye-quencher pair is FAM, which emits green light, and MGB-NFQ (minor groove binding non fluorescent quencher). According to exemplary embodiments of the present invention, the fluorescent reporter dye-quencher pair is VIC and MGB-NFQ. Another example of a fluorescent reporter dye-quencher pair is FAM and Black Hole Quencher 1 dye. Another example of a fluorescent reporter dye-quencher pair is VIC and BHQ-1. Selection of fluorescent reporter dye-quencher pairs is discussed in more detail in Maras, Salvatore AE. Selection of fluorophore and quencher pairs for fluorescent nucleic acid hybridization probes, Fluorescent Energy Transfer Nucleic Acid Probes. Humana Press, 2006. 3-16.

### Hybridization Probe Specificity

Each of the one or more hybridization probes are complementary to a variable region (i.e. a region that is not conserved) between ELS and non-ELS cotton species (see, e.g., FIG. 1). For example and without limitation, a first hybridization probe is complementary to a first specific sequence of a variable region of G. barbadense and a second hybridization probe is complementary to a second specific sequence of a variable region of G. hirsutum. According to an exemplary embodiment of the present invention, the variable regions are in non-conserved regions of chloroplast DNA of G. barbadense and G. hirsutum.

By comparing DNA sequences of G. barbadense (GenBank Accession No. NC_{—}008641) and G. hirsutum (GenBank Accession No. NC_{—}007944), variable regions between the two species were detected and possible genetic markers to distinguish between these two cotton species were identified in the variable regions.

According to exemplary embodiments of the present invention, the variable regions may include a sequence polymorphism between the first cotton species and the second cotton species of the one or more cotton species. The sequence polymorphism may include one or more single nucleotide polymorphisms (SNPs). The sequence polymorphism may include a sequence length polymorphism. The sequence polymorphism may include one or more nucleotide insertions or deletions. The variable region may include a sequence length polymorphism between the first cotton species and the second cotton species. For example and without limitation, the sequence length polymorphism includes one or more short tandem repeats (STRs). The variable region may include one or more microsatellites, which are also referred to as simple sequence repeats (SSRs).

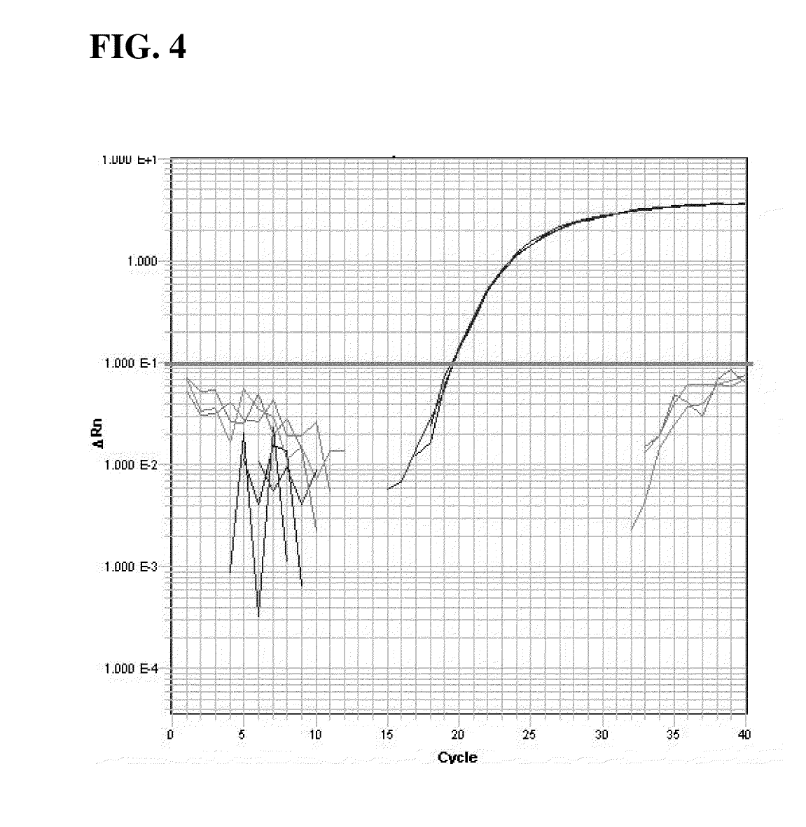

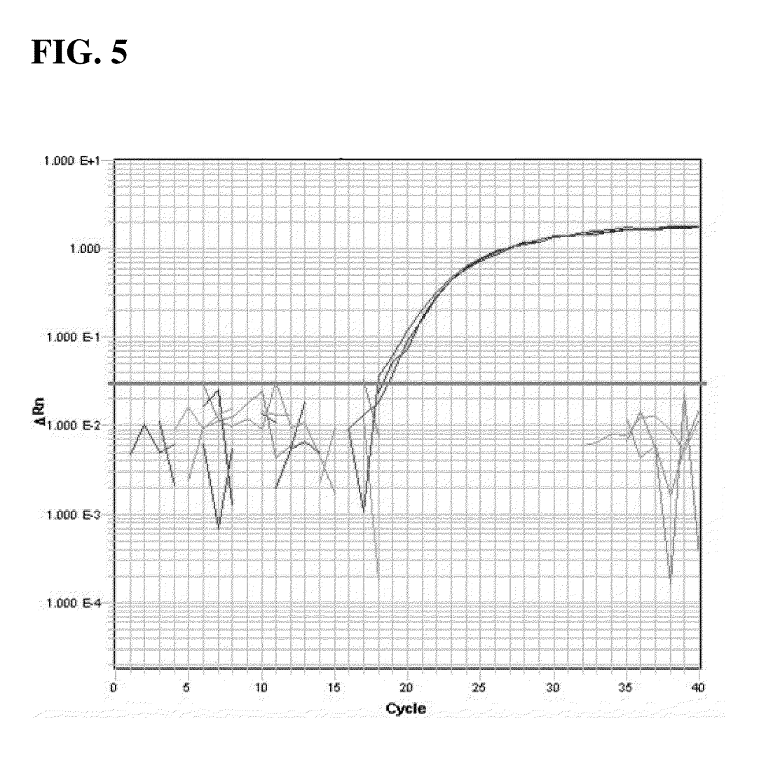

Referring to FIGS. 4 and 5, the probes were tested for specificity for ELS and Upland cotton, respectively. The ELS probe was tested against Upland DNA, and Upland probe was tested against ELS DNA. The ELS probe did not detect Upland DNA and the Upland probe did not detect ELS DNA.

FIG. 4 is a multiplex qPCR amplification curve for a sample from a textile article including 100% ELS cotton. With reference to the multiplex qPCR curve of FIG. 4, the reaction included a first fluorescently labeled hybridization probe complementary to a variable region of ELS cotton (cultivar E503, illustrated below) and a second hybridization probe complementary to a variable region of non-ELS cotton (cultivar CPCSD Acala Daytona RF, illustrated below). However, the sample was obtained from a textile article including 100% ELS cotton. In the multiplex qPCR amplification curve for the 100% ELS sample, only an ELS (cultivar E503) amplification curve was observed. The Upland cultivar (cultivar CPCSD Acala Daytona RF) did not produce a detectable amplification curve.

FIG. 5 is a multiplex qPCR amplification curve for a sample from a textile article including 100% non-ELS cotton. With reference to the multiplex qPCR curve of FIG. 5, the reaction included a first fluorescently labeled hybridization probe complementary to a variable region of ELS cotton (cultivar E503, illustrated below) and a second hybridization probe complementary to a variable region of non-ELS cotton (cultivar CPCSD Acala Daytona RF, illustrated below). However, the sample was obtained from a textile article including 100% non-ELS (Upland) cotton. In the multiplex qPCR amplification curve for the 100% Upland sample, only an Upland (cultivar CPCSD Acala Daytona RF) amplification curve was observed. The ELS (cultivar E503) cultivar did not produce a detectable amplification curve.

### Cultivar Coverage

Cotton species, including G. barbadense and G. hirsutum, each include a plurality of cotton cultivars. Several cultivars from each species were tested and all of the cultivars were identified correctly by the methods according to exemplary embodiments of the present invention as ELS or Upland, respectively. No cross reactions were detected between the hybridization probe specific for Upland cotton and the hybridization probe specific for ELS cotton.

TABLE 1

| Species | Exemplary Cultivars | | | | | | |
|---|---|---|---|---|---|---|---|
| ELS | DP353 | E503 | OA353 | PO3X8161 | Egyp- | ELS- | — |
| | | | EXP | | tian | Chinese | |
| | | | | | Giza | | |
| | | | | | 86 | | |
| Upland | CPCSD | CPCSD | PHY72 | Phytogen | Delta | Lint | Lint |
| | Acala | Acala | Acala | PHY725RF | & | 444 | 445 |
| | Fiesta | Daytona | | | pine | | |
| | RR | RF | | | | | |

### Quantitative Analysis of Cotton DNA

Cotton DNA may be quantitatively analyzed to assess a proportion of one or more cotton species included in the article including cotton from which the cotton fibers were obtained. For example, the cotton DNA may be amplified by qPCR to determine a threshold cycle number for the extracted cotton DNA of each identified cotton species in a sample including cotton.

### Threshold Cycles

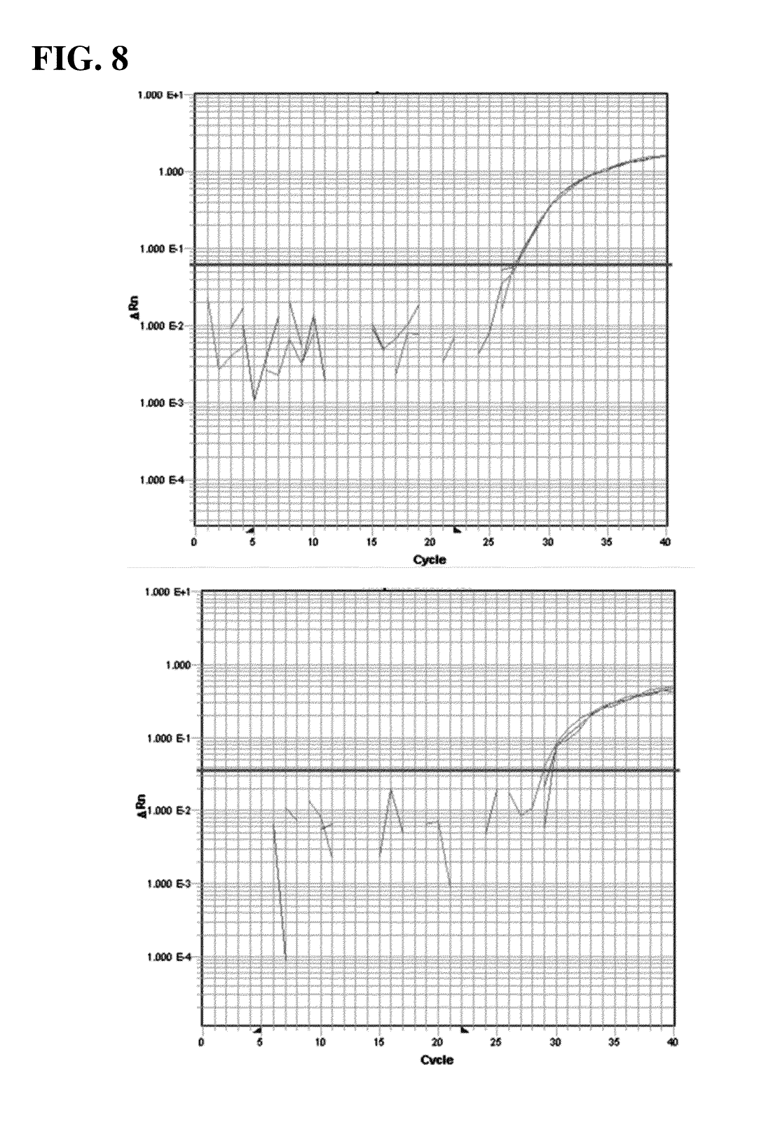

A threshold cycle number represents the number of PCR cycles a particular amount of a DNA template must undergo in order to surpass a minimum threshold amplification level. The minimum threshold amplification level may be automatically determined by the qPCR instrument performing the qPCR amplification. Determining the threshold cycle number for a particular DNA sample may be used to determine the relative amount of DNA template included in a DNA sample. For example and without limitation, a threshold cycle number is determined for a provided DNA sample and the threshold cycle number is compared to a known standard to determine the amount of DNA template provided in the DNA sample. As illustrated in FIGS. 8 to 10, threshold cycle numbers will be higher when the amount of DNA in a sample is relatively small, and threshold cycle numbers will be lower when the amount of DNA in a sample is relatively large.

According to exemplary embodiments of the present invention, the threshold cycle number is determined for the cotton DNA extracted from the cotton fibers of the article including cotton. The threshold cycle numbers are determined for extracted cotton DNA for each cotton species identified in the article including cotton. The threshold cycle numbers are determined by qPCR amplification. The threshold cycle number for a cotton species are compared to a known threshold cycle number to assess a proportion of the cotton species included in the article including cotton.

According to exemplary embodiments of the present invention, an article including cotton includes a first cotton species and a second cotton species. The first and second cotton species are identified as being included in the article including cotton. A first threshold cycle number for the extracted cotton DNA of the first cotton species and a second threshold cycle number for the extracted cotton DNA of the second cotton species are determined. The first threshold cycle number is compared to the second threshold cycle number and proportions of the first and second cotton species included in the article including cotton are thereby assessed. For example and without limitation, an article including cotton includes a blend of G. barbadense and G. hirsutum cotton. The first threshold cycle number is determined for extracted cotton DNA of G. barbadense and the second threshold cycle number is determined for extracted cotton DNA of G. hirsutum and proportions of G. barbadense and G. hirsutum included in the article including cotton are assessed. For example, assessing proportions of G. barbadense and G. hirsutum cotton may determine that the article including cotton includes a blend of 80% G. barbadense and 20% G. hirsutum cotton. In the case of a blend of 80% G. barbadense and 20% G. hirsutum cotton, the amount of cotton DNA extracted from the G. barbadense cotton fibers would be relatively greater than the amount of cotton DNA extracted from the G. hirsutum cotton fibers. Therefore, the threshold cycle number for the cotton DNA extracted from the G. barbadense cotton fibers would be relatively low and the threshold cycle number for the cotton DNA extracted from the G. hirsutum cotton fibers would be relatively high.

Method Accuracy with Various Blends

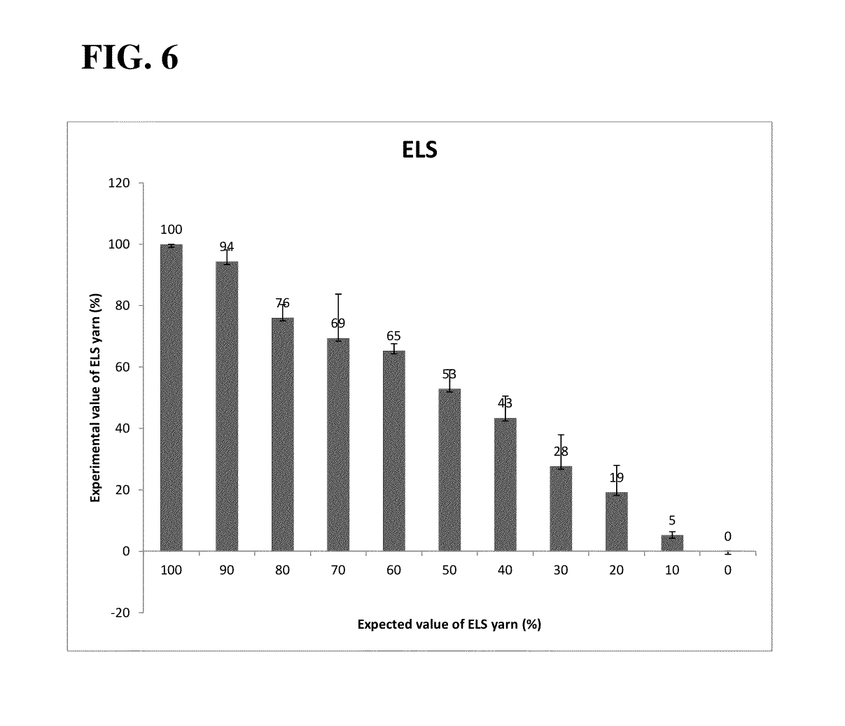

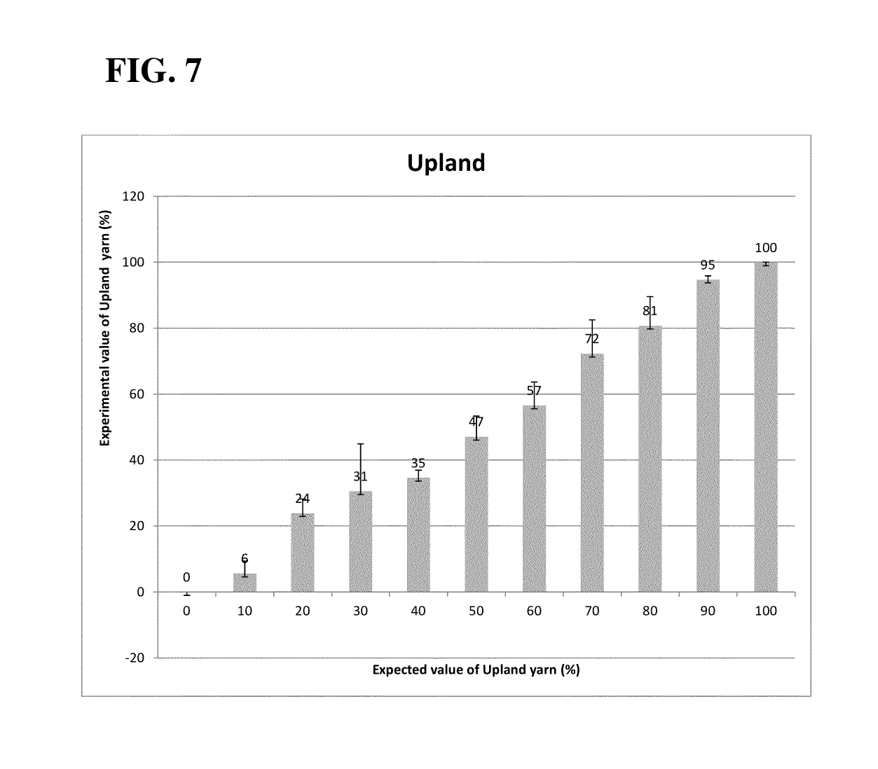

FIG. 6 is a graph illustrating experimentally determined proportions of ELS cotton included in an article including cotton compared with known proportions of ELS cotton included in the article including cotton. FIG. 7 is a graph illustrating experimentally determined proportions of non-ELS cotton included in an article including cotton compared with known proportions of ELS cotton included in the article including cotton. Referring to FIGS. 6 and 7, various blends were tested using the method according to exemplary embodiments of the present invention. Cotton blends of known purity were tested. The cotton blends tested ranged from 100% ELS cotton to 0% ELS cotton. Cotton blends that were not 100% ELS cotton included a known corresponding proportion of Upland cotton. FIGS. 6 and 7 illustrate the Experimental values on the Y axis and the expected (known) values of ELS and upland cotton, respectively, on the X axis. Error bars are illustrated in FIGS. 6 and 7. The error bars were determined based on the standard deviations of the experimental values.

Assessing Proportions of Cotton Species by qPCR Amplification

According to exemplary embodiments of the present invention, a portion of the cotton DNA extracted from the cotton fibers of the article including cotton is amplified. The portion of the extracted cotton DNA is amplified by qPCR and one or more amplified products (e.g., amplicons) are produced. The amplified portion of the extracted cotton DNA is amplified by using chloroplast DNA as a template.

qPCR amplification of the extracted cotton DNA may be performed singlplex or multiplex. In multiplex qPCR, multiple portions of the extracted cotton DNA are amplified in a single reaction tube. Each portion of the extracted cotton DNA is amplified by the specific set of primers, and the unique hybridization probes are used to identify each cotton species included in the article including cotton. In singleplex qPCR the portions of the cotton DNA extracted from the cotton fibers are amplified in separate reaction tubes and the results are compared.

According to an exemplary embodiment of the present invention, portions of variable regions of ELS and Upland cotton DNA extracted from cotton fibers are amplified by multiplex qPCR. A single set of forward and reverse primers are complementary to non-variable regions of both cotton species and therefore a single set of primers can amplify DNA from both species. The hybridization probes (i.e., probes which are complementary to the variable regions between ELS and Upland cotton) are used to distinguish ELS from Upland DNA.

FIG. 8 illustrates multiplex qPCR amplification curves showing threshold cycle numbers for ELS and Upland cotton included in a textile article including a blend of ELS and Upland cotton. FIG. 8 illustrates a Multiplex qPCR amplification curve of 80% ELS and 20% Upland blended yarn. The curve on the top illustrates ΔRn for an ELS probe. The curve on the bottom illustrates ΔRn for an Upland probe. All lines are shown at triplicate.

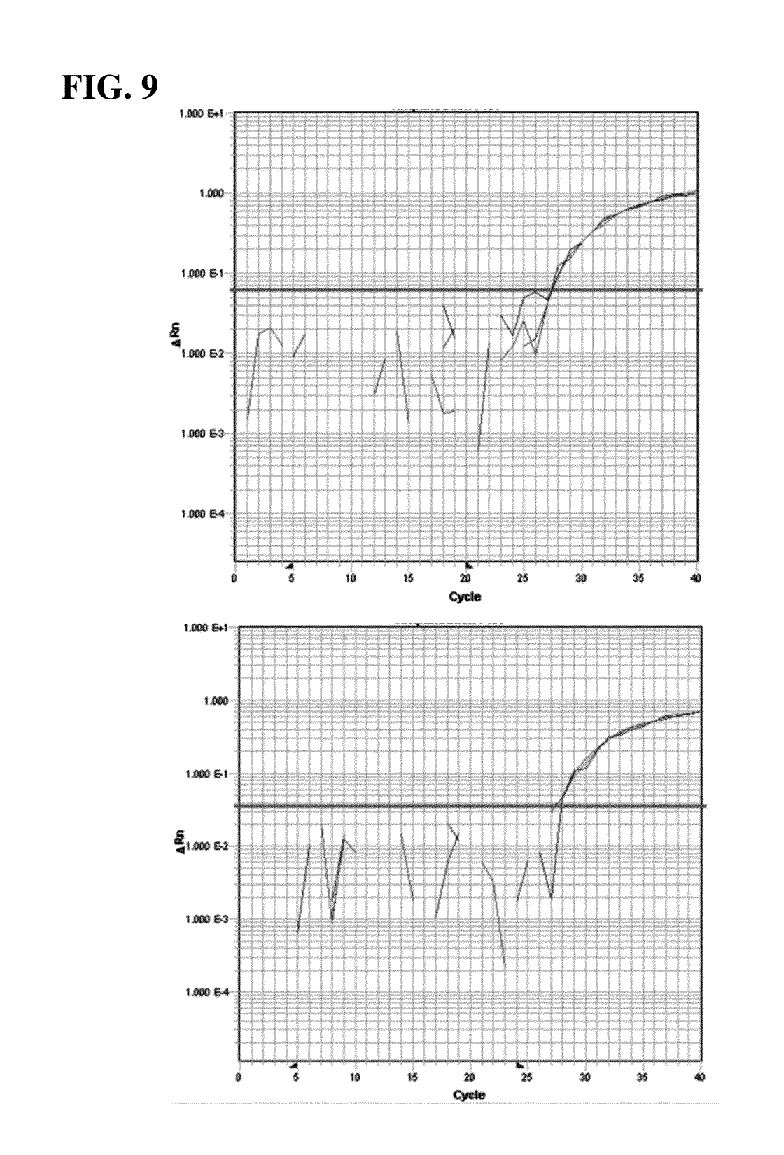

FIG. 9 illustrates multiplex qPCR amplification curves showing threshold cycle numbers for ELS and Upland cotton included in a textile article including a blend of ELS and Upland cotton. FIG. 9 illustrates a Multiplex qPCR amplification curve of 50% ELS and 50% Upland blended yarn. The curve on the top illustrates ΔRn for an ELS probe. The curve on the bottom illustrates ΔRn for an Upland probe. All lines are shown at triplicate.

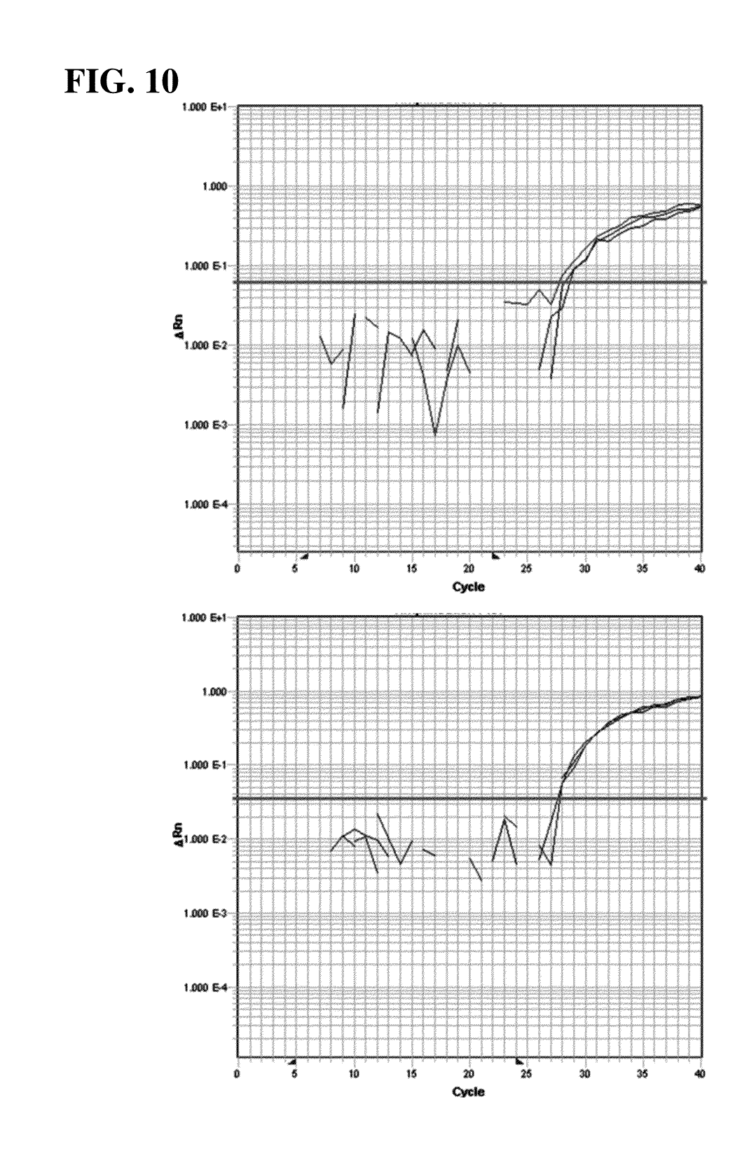

FIG. 10 illustrates multiplex qPCR amplification curves showing threshold cycle numbers for ELS and Upland cotton included in a textile article including a blend of ELS and Upland cotton. FIG. 10 illustrates a Multiplex qPCR amplification curve of 20% ELS and 80% Upland blended yarn. The curve on the top illustrates ΔRn for an ELS probe. The curve on the bottom illustrates ΔRn for an Upland probe. All lines are shown at triplicate.

With reference to FIGS. 8-10, Rn refers to the fluorescence of the reporter dye divided by a fluorescence of a passive reference dye (e.g., a baseline). ΔRn refers to Rn minus the baseline fluorescence. ΔRn or log (ΔRn) may be plotted against PCR cycle number. The amplification curves illustrated in FIGS. 8-10 illustrate the variation of log (ΔRn) plotted against the PCR cycle number.

Example of DNA Extraction and qPCR Preparation

A textile article including mature cotton fibers was provided. The mature cotton fibers were collected from the textile article and a sample including the mature cotton fibers was prepared. The sample was weighed and prepared for DNA extraction. The sample weighed from about 10 mg to about 15 mg.

The sample was transferred to a 1.5 ml eppendorf tube for DNA extraction and qPCR analysis. 200 μL of Extraction Buffer (Part #E7526, Sigma-Aldrich was added to the eppendorf tube containing the sample. The Extraction Buffer and the sample were incubated at 95° C. for 30 minutes. After 30 minutes 200 μL of Dilution Buffer (Part #D5688, Sigma-Aldrich) was added to the Extraction Buffer. The Dilution Buffer was added to stop the reaction between the Extraction Buffer and the sample. The eppendorf was then vortexed until the contents were well mixed. The extracted solution was used as a PCR template for qPCR.

A 96 well plate was prepared for qPCR. Each well of the 96 well plate included a 200 μl reaction mixture. The 20 μl reaction mixture included 10 μl TaqMan® Fast Advanced Master Mix (Part #4444602, Life Technologies), 0.50 of 10 uM forward primer (5′-AAT CCC AGG GAA ATA AAG AAA AGT GTA-3′) 0.50 of 10 uM reverse primer (5′-TTA CAA CCC GGC TTC GAA TCT A-3′), 0.50 of 10 uM ELS probe (5′-6FAM-ATG ATT TCA TTC AAG CCA TTT-MGBNFQ-3′ (Part#4316033, Life Technologies), 0.50 of 10 uM Upland probe (5′-VIC-TCT TAT GAT TTC ATT CAT TTT C-MGBNFQ-3′ (Part#4316033, Life Technologies), 20 μl of PCR template (including DNA extracted from the sample) and 60 μl of water. The 96 well plate was then loaded into a qPCR instrument for analysis (ABI 7900HT, Life Technologies). qPCR was performed with the following cycling parameters: 50° C. for 2 minutes, followed by 95° C. for 20 seconds, then 40 cycles of 95° C. for 1 seconds, and 60° C. for 20 seconds. Data was analyzed by SDS software (Life Technologies) and proportions of each species of cotton included in the sample were determined.

The disclosures of each of the references, patents and published patent applications disclosed herein are each hereby incorporated by reference herein in their entireties.

In the event of a conflict between a definition herein and a definition incorporated by reference, the definition provided herein is intended.

Having described exemplary embodiments of the present invention, it is further noted that it is readily apparent to those of ordinary skill in the art that various modifications may be made without departing from the spirit and scope of the present invention.

What is claimed is:

1. A method for assessing a proportion of one or more cotton species in an article comprising cotton, the method comprising:
providing a sample including cotton fibers from the article comprising cotton;
extracting cotton DNA from the cotton fibers to provide extracted cotton DNA;
analyzing the extracted cotton DNA and thereby identifying a presence of one or more cotton species included in the article comprising cotton; and
assessing the proportion of the one or more cotton species included in the article comprising cotton.

2. The method of claim 1, wherein the extracted cotton DNA comprises chloroplast DNA.

3. The method of claim 1, wherein the one or more cotton species comprises G. barbadense.

4. The method of claim 1, wherein the one or more cotton species comprises G. hirsutum.

5. The method of claim 1, wherein analyzing the extracted cotton DNA comprises a polymerase chain reaction (PCR).

6. The method of claim 1, wherein analyzing the extracted cotton DNA comprises amplifying the extracted cotton DNA using at least one set of specific primers complementary to a non-variable region of the one or more cotton species.

7. The method of claim 1, wherein the one or more cotton species are identified by using one or more hybridization probes each complementary to a variable region sequence specific to a cotton species of the one or more cotton species.

8. The method of claim 7, wherein the variable region sequence specific to the cotton species of the one or more cotton species is in a variable region of G. barbadense.

9. The method of claim 7, wherein the variable region sequence specific to the cotton species of the one or more cotton species is in a variable region of G. hirsutum.

10. The method of claim 7, wherein the hybridization probe comprises a detectable marker.

11. The method of claim 10, wherein the sequence specific to the cotton species of the one or more cotton species comprises a sequence polymorphism between a first cotton species and a second cotton species of the one or more cotton species, and wherein the sequence polymorphism between the first cotton species and the second cotton species does not include a length polymorphism.

12. The method of claim 10, wherein the sequence specific to the cotton species of the one or more cotton species comprises a sequence length polymorphism between a first cotton species and a second cotton species of the one or more cotton species.

13. The method of claim 1, wherein the extracted cotton DNA comprises nuclear DNA and/or mitochondrial.

14. A method for assessing a proportion of one or more cotton species in an article comprising cotton, the method comprising:
providing a sample including cotton fibers from the article comprising cotton;
extracting cotton DNA from the cotton fibers to provide extracted cotton DNA;
amplifying a portion of the extracted cotton DNA by qPCR producing one or more amplified products;
analyzing the one or more amplified products and thereby identifying a presence of at least one cotton species in the cotton fibers from the article comprising cotton;
determining a threshold cycle number for the extracted cotton DNA;
comparing the threshold cycle number for the extracted cotton DNA to a known threshold cycle number; and
assessing the proportion of the cotton species included in the article comprising cotton.

15. The method of claim 14, wherein the amplified portion of the extracted cotton DNA is amplified from chloroplast DNA.

16. The method of claim 14, wherein the one or more cotton species comprises G. barbadense.

17. The method of claim 14, wherein the one or more cotton species comprises G. hirsutum.

18. A method for assessing a proportion of one or more cotton species in an article comprising cotton, the method comprising:
providing a sample including cotton fibers from the article comprising cotton;
extracting DNA from the cotton fibers to provide extracted cotton DNA;
amplifying a portion of the extracted cotton DNA by qPCR producing one or more amplified products;
analyzing the one or more amplified products and thereby identifying a presence of at least a first cotton species and/or a second cotton species in the cotton fibers from the article comprising cotton;
determining a first threshold cycle number for extracted cotton DNA of the first cotton species and a second threshold cycle number for extracted cotton DNA of the second cotton species;
comparing the first and second threshold cycle numbers; and
assessing proportions of the first cotton species and the second cotton species included in the article comprising cotton.

19. The method of claim 18, wherein the one or more cotton species comprises G. barbadense.

20. The method of claim 18, wherein the extracted cotton DNA is amplified by multiplex qPCR.


SEQUENCE LISTING (4 sequences)

SEQ ID NO: 1 - DNA, 27 bp, Artificial Sequence
  Forward Primer for Gossypium Barbadense or Gossypium Hirsutum
aatcccagggaaataaagaaaagtgta

SEQ ID NO: 2 - DNA, 22 bp, Artificial Sequence
  Reverse Primer for Gossypium Barbadense or Gossypium Hirsutum
ttacaacccggcttcgaatcta

SEQ ID NO: 3 - DNA, 21 bp, Artificial Sequence
  ELS Cotton Probe Sequence
atgatttcattcaagccattt

SEQ ID NO: 4 - DNA, 22 bp, Artificial Sequence
  Upland Cotton Probe Sequence
tcttatgatttcattcattttc